(19) United States
(12) Patent Application Publication
(10) Pub. No.: US 2026/0224239 A1
(43) Pub. Date: Aug. 6, 2026

(54) INTRAVASCULAR SCRAPING DEVICES

(71) Applicant: Bard Peripheral Vascular, Inc., Franklin Lakes, NJ (US)

(72) Inventors: Andrzej J. Chanduszko, Chandler, AZ (US); Richard L. Abbott, Raleigh, NC (US); Chad C. Van Liere, Phoenix, AZ (US)

(21) Appl. No.: 19/046,237

(22) Filed: Feb. 5, 2025

(51) Int. Cl. A61B 17/3207; A61B 17/00; A61B 17/32

(52) U.S. Cl. CPC A61B 17/3207; A61B 2017/00526; A61B 2017/320008

(57) ABSTRACT

An example intravascular scraping device (“ISD”) includes a proximal end configured to be attached to a catheter of a catheter system. The ISD may optionally include a distal end spaced from the proximal end that is configured to be attached to the catheter. The ISD includes a plurality of cutting segments, each of the plurality of cutting segments include one or more cutting elements that are oriented radially outward from the catheter. The ISD further includes a plurality of linking segments, at least some of which link the cutting segments to the proximal end. The linking segments may be integrally formed (e.g., exhibit single piece construction) with or attached to at least one of the proximal end or the cutting segments.

### BACKGROUND

Conventional thermal ablation technology, such as radio frequency and laser thermal ablation, can require tumescent anesthesia and are associated with intra-and post-procedure pain, bruising, and discomfort. Non-thermal, non-tumescent treatment portions include mechanochemical ablation.

An example of a conventional mechanochemical ablation device includes an infusion catheter with a rotating angled wire tip designed to disperse liquid sclerosant. The tip of this conventional mechanochemical ablation device is relatively atraumatic and, therefore, does not produce any significant damage to venous walls.

### SUMMARY

Embodiments disclosed herein related to intravascular scraping devices (“ISD”), catheter systems including the same, and methods of making and using the same. In an embodiment, an intravascular scraping device (“ISD”) is disclosed. The ISD includes a proximal end configured to be attached to a catheter. The ISD also includes a plurality of cutting segments. Each of the plurality of cutting segments includes one or more cutting elements. Additionally, the ISD includes a plurality of linking segments. At least some of the plurality of linking segments link the plurality of cutting segments to the proximal end. At least some of the plurality of linking segments directly extend from the proximal end and at least some of the plurality of linking segments directly extend from the plurality of cutting segments generally towards the proximal end.

In an embodiment, a catheter is disclosed. The catheter includes a central catheter including a distal end region. The catheter also includes an ISD. The ISD includes a proximal end configured to be attached to a first portion of the distal end region. The ISD also includes a plurality of cutting segments. Each of the plurality of cutting segments includes one or more cutting elements. Additionally, the ISD includes a plurality of linking segments. At least some of the plurality of linking segments link the plurality of cutting segments to the proximal end. At least some of the plurality of linking segments directly extend from the proximal end and at least some of the plurality of linking segments directly extend from the plurality of cutting segments generally towards the proximal end.

In an embodiment, a method of forming an ISD is disclosed. The method includes providing at least one generally cylindrical tube and cutting the generally cylindrical tube to form at least a proximal end configured to be attached to a catheter, a plurality of cutting segments, and a plurality of linking segments linking each of the plurality of cutting segments to at least the proximal end. Each of the plurality of cutting segments includes one or more cutting elements. At least some of the plurality of linking segments directly extend from the proximal end and at least some of the plurality of linking segments directly extend from the plurality of cutting segments generally towards the proximal end.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

FIG. 1A is a schematic side view of a catheter system, according to an embodiment.

FIG. 1B is an isometric view of the ISD in the expanded configuration, according to an embodiment.

FIG. 1C is a top plan view of a cutting element, according to an embodiment.

FIG. 2 is an isometric view of an ISD, according to an embodiment.

FIGS. 3A and 3B are isometric views of an unassembled ISD and an assembled ISD, respectively, and illustrate a method of forming the assembled ISD, according to an embodiment.

FIGS. 4A and 4B are isometric views of an unassembled ISD and an assembled ISD, respectively, and illustrate a method of forming the assembled ISD, according to an embodiment.

FIGS. 5A and 5B are an isometric and side plan view of an ISD, according to an embodiment.

The drawings illustrate several embodiments of the present disclosure, wherein identical reference numerals refer to identical or similar elements or features in different views or embodiments shown in the drawings.

### DETAILED DESCRIPTION

Embodiments disclosed herein related to intravascular scraping devices (“ISD”), catheter systems including the same, and methods of making and using the same. An example ISD includes a proximal end configured to be attached to a catheter of a catheter system. The ISD may, in some embodiments, include a distal end spaced from the proximal end that is configured to be attached to the catheter. The ISD includes a plurality of cutting segments, each of the plurality of cutting segments include one or more cutting elements that are oriented radially outward from the catheter. The ISD further includes a plurality of linking segments, at least some of which link the cutting segments to the proximal end. The linking segments may be integrally formed (e.g., exhibit single piece construction) with or attached to at least one of the proximal end or the cutting segments.

The ISD may be attached to a catheter of a catheter system. During use, the ISD may initially exhibit a collapsed configuration. The ISD exhibits a collapsed configuration when a maximum lateral dimension (e.g., diameter) of the ISD is sufficiently small to be inserted into the vascular structure. The ISD may be configured to prevent or at least inhibit the cutting elements of the ISD from damaging the vascular structure. With the ISD in the collapsed configuration, the catheter may be inserted into the individual. The catheter may be advanced (e.g., moved) in the individual until the ISD is positioned in or adjacent to a desired vascular structure. The desired vascular structure may include, for example, a varicose vein. The ISD may switch from the collapsed configuration to an expanded configuration, which includes increasing the maximum lateral dimension until the cutting elements of the ISD contact a surface and, optionally, protrude slightly into the vascular structure. The catheter and the ISD may then move relative to or within the vascular structure thereby causing the cutting elements to scrape against and damage one or more surfaces of the vascular structure. Such movement of the catheter and the ISD may include a pulling movement (e.g., moving the ISD towards the incision that allowed the catheter and the ISD to enter the individual), a pushing movement (e.g., moving the ISD away from the incision), rotating the catheter and ISD in the vascular structure, or combinations thereof. The damage to the vascular structure caused by the cutting elements may promote collapse of the vascular structure or another beneficial or desired effect. In an embodiment, the catheter may also inject a sclerosant or other chemical or drug into the vascular structure before, during, or after moving the ISD in the vascular structure. The damage caused by the cutting elements may improve the effect of the sclerosant or other chemical or drug.

The ISD disclosed herein may be an improvement over other conventional devices that are configured to damage one or more surfaces of a vascular structure (“conventional devices”). An example conventional device includes thermal ablation devices that use radio frequencies and/or lasers to damage one or more surfaces of a vascular structure. The thermal ablation devices are used in a vast majority of varicose vein procedures. However, conventional thermal ablation devices require the use of tumescent anesthesia and can cause pain intra-procedure and post-procedure.

Non-thermal, non-tumescent treatment devices attempt to solve several of these issues associated with conventional thermal ablation devices. Examples of non-thermal, non-tumescent treatment devices include mechanochemical ablation devices. Mechanochemical ablation devices rely on mechanical and/or chemical means (e.g., sclerosant) to damage the surfaces of the vascular structure. Conventional mechanochemical ablation devices have several issues associated thereof. In an example, conventional mechanochemical ablation devices that use mechanical means to damage the walls of the vascular structure often apply too great of pressure against the vascular structure which can cause injury to the vascular structure, injury to the surrounding tissue if the pressure allows the mechanochemical ablation device to puncture through the vascular structure, cause intra-procedural and post-procedural pain, and increase the risk of infection. Also, conventional mechanochemical ablation devices that use mechanical means to damage the walls of the vascular structure often apply inconsistent pressure against the vascular structure. The inconsistent pressure may cause the vascular structure to be relatively undamaged in portions of the vascular structure that had too little pressure applied thereto while other portions of the vascular structure may have too great of pressure applied to the vascular structure. Some conventional mechanochemical ablation devices attempt to avoid these issues associated with mechanochemical ablation devices that use mechanical means to damage the walls by only applying chemicals to the vascular structure. The effectiveness of such conventional mechanochemical ablation devices may be limited to the effectiveness of the chemical unlike the mechanochemical ablation devices that use mechanical means to damage the walls since the mechanical means enhance the effectiveness of the chemical means.

The ISDs disclosed herein solve these issues associated with conventional mechanochemical ablation devices. For example, the ISDs disclosed herein include structures and/or materials that allow the ISDs to apply a more consistent pressure against the vascular structures during use thereby preventing or at least inhibiting the issues associated with applying too great of pressure or inconsistent pressure against the vascular structure. The ISDs disclosed herein also include cutting elements that damage the walls of the vascular structure thereby enhancing the effect of the chemicals dispensed from the ISDs or the catheter to which the ISDs are attached.

Although the ISDs disclosed herein are often discussed as being used in varicose vein procedures, it is noted that the ISDs disclosed herein may be used in other procedures. In an example, the ISDs disclosed herein may be used to induce vessel spasms prior to vein closure treatments with adhesives or the use of thermal ablation devices. Inducing such spasms may reduce the diameter of the vascular structure thereby making other treatments easier or more effective (e.g., large vascular structure conventionally require multiple treatments in a single procedure or across multiple procedures due to vessel reopening). In an example, the ISDs disclosed herein may be used in valvulotome procedures, including peripheral bypass or coronary artery bypass procedures, to disable venous valves.

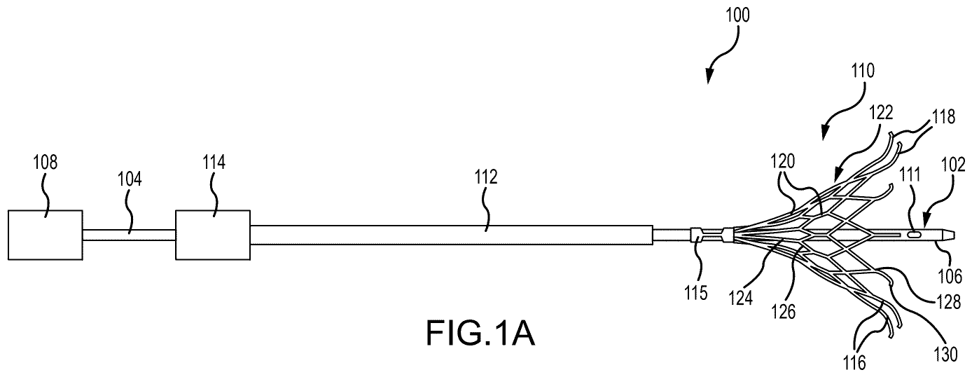

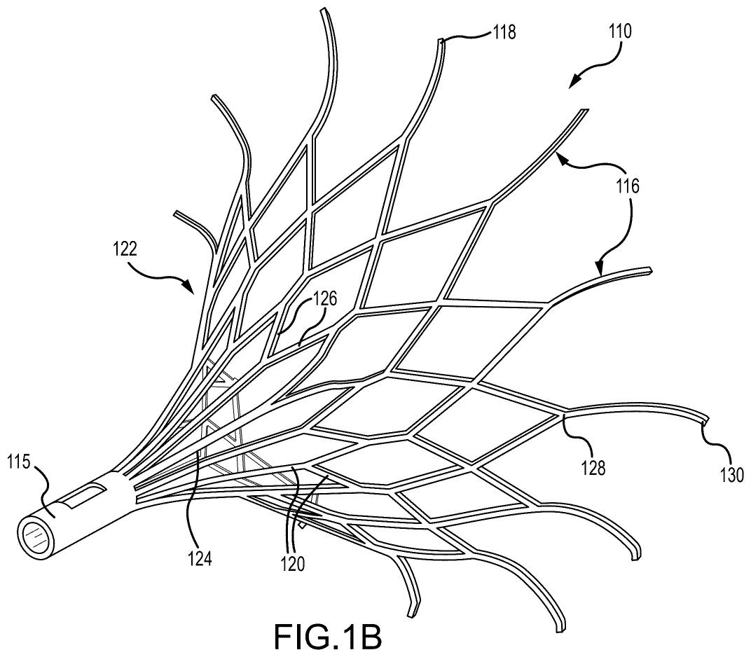

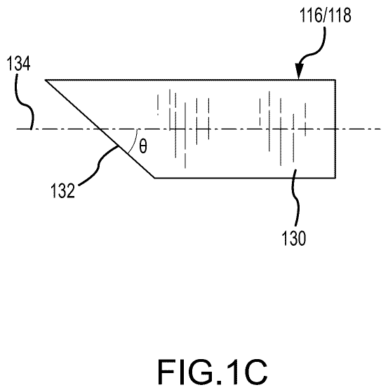

FIG. 1A is a schematic side view of a catheter system 100, according to an embodiment. The catheter system 100 includes a catheter 102. The catheter 102 may include a proximal end region 104 and a distal end region 106. The proximal end region 104 may be attached to a handle 108 and the distal end region 106 may include an ISD 110 attached thereto. The handle 108 may be configured to move the catheter 102 and the ISD 110 when the catheter 102 and the ISD 110 are inserted into an individual. For example, moving the handle 108 in a distal direction (e.g., in a direction extending from the proximal end region 104 to the distal end region 106) may advance the catheter 102 and the ISD 110 into an individual and moving the handle 108 in a proximal direction (e.g., in a direction extending from the distal end region 106 to the proximal end region 104) may retract the catheter 102 from the individual and pull on the ISD 110. It is noted that the handle 108 may exhibit any structure known in the art and may move the catheter 102 and the ISD 110 using any conventional technique.

In an embodiment, the catheter 102 may define one or more holes 111. For example, the holes 111 may be defined by the distal end region 106. The holes 111 may be configured to dispense sclerosants or other chemicals during use of the catheter system 100. For example, the catheter 102 may define one or more passageways (not shown) in fluid communication with the holes 111. The passageways may be configured to provide the sclerosants or other chemicals to the holes 111 from a source of the sclerosants or other chemicals. The sclerosants or other chemicals may be selected to facilitate operation of the catheter system 100. For instance, when the catheter system 100 is configured to promote collapse of the vascular structure, the sclerosants or other chemicals may be selected to also promote collapse of the vascular structure. It is noted that the mechanical damage caused by the ISD 110 may improve the efficacy of the sclerosants or other chemicals.

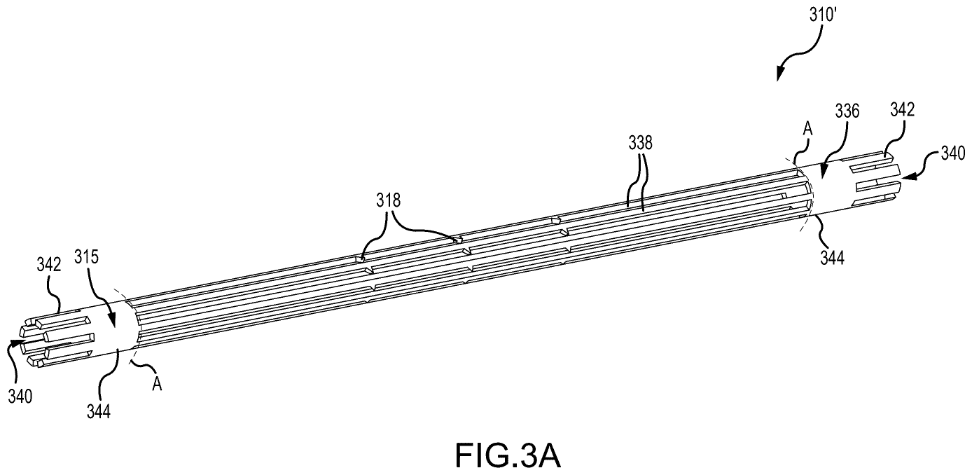

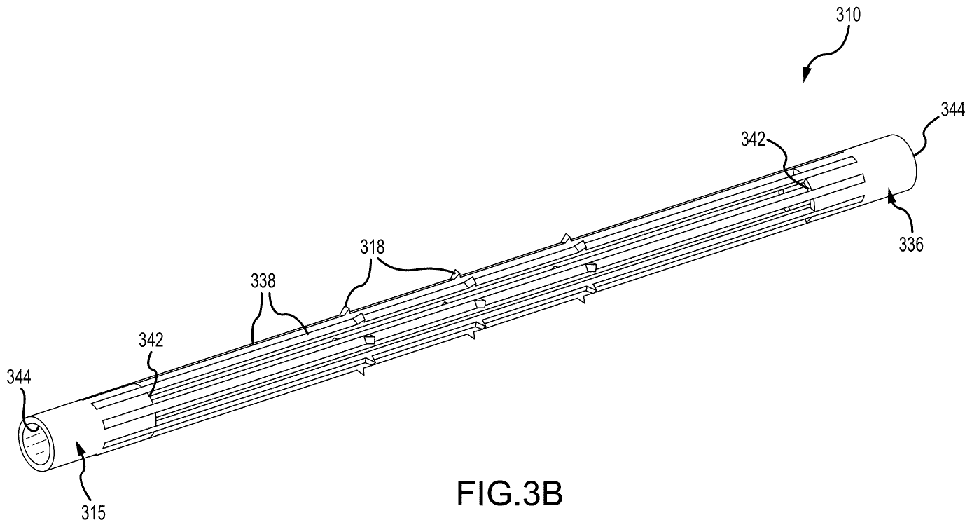

The catheter system 100 may also include a sheath 112 configured to switch the ISD 110 between a collapsed configuration (an ISD in the collapsed configuration is shown in FIG. 3B) and an expanded or deployed configuration (shown in FIG. 1A). The sheath 112 is configured to slide along the catheter 102. Sliding the sheath 112 in a distal direction may cause the sheath 112 to receive the ISD 110. Receiving the ISD 110 into the sheath 112 may cause the ISD 110 to be in the collapsed configuration by forcing the maximum lateral dimension of the ISD 110 to decrease until the maximum lateral dimension of the ISD 110 is small enough to fit within the sheath 112. For example, the ISD 110 is radially collapsible to a smaller diameter to be contained at least partially within the sheath 112 for insertion and retrieval. Sliding the sheath 112 in a proximal direction may cause the sheath 112 to expose the ISD 110, thereby allowing the ISD 110 to switch from the collapsed configuration to the expanded configuration. Exposing the ISD 110 allows the maximum lateral dimension of the ISD 110 to increase. In an embodiment, the catheter system 100 may include an actuator 114 that, when actuated, causes the sheath 112 to move in the distal direction or the proximal direction. The actuator 114 may be distinct (e.g., spaced) from the handle 108 (as shown) or form part of the handle 108.

FIG. 1B is an isometric view of the ISD 110 in the expanded configuration, according to an embodiment. The ISD 110 includes a proximal end 115 configured to be attached to the catheter 102. The ISD 110 also includes a plurality of cutting segments 116. Each of the cutting segments 116 includes a cutting element 118. The cutting elements 118 are generally oriented radially outward relative to the catheter 102. The ISD 110 also includes a plurality of linking segments 120 linking each of the cutting segments 116 to the proximal end 115. Some of the linking segments directly extend from the proximal end 115 and some of the linking segments 120 directly extending from the cutting segments 116 generally towards the proximal end 115.

The proximal end 115 of the ISD 110 is at or near the proximal terminal end of the ISD 110 (i.e., the end of the ISD 110 facing the proximal direction). The proximal end 115 is a portion of the ISD 110 that is configured to be attached to the catheter 102, thereby securing the other components to the catheter 102. The proximal end 115 may be attached to the catheter 102 using any suitable technique. In an example, the proximal end 115 may be crimped to the catheter 102. In an example, the proximal end 115 may be attached to the catheter 102 using a weld, an adhesive, press-fit, mechanical interlocking, or any other suitable technique.

As previously discussed, the ISD 110 includes a plurality of linking segments 120. The linking segments 120 extend between and link the proximal end 115 and the cutting segments 116 together. As such, the linking segments 120 indirectly secure the cutting segments 116 to the proximal end 115. In some embodiments, the linking segments 120 may also extend between and link together adjacent ones of the linking segments 120 thereby forming an interconnected structure 122.

During use, the interconnected structure 122 formed by the linking segments 120 is configured to abut against one or more inner surfaces of the vascular structure when the ISD 110 is in the expanded configuration. Allowing the interconnected structure 122 to abut against the inner surface of the vascular structure which allows the cutting elements 118 to apply a consistent and gentle pressure against the inner surfaces of the vascular structure. Abutting the interconnected structure 122 against the inner surface of the vascular structure allows the lateral dimensions of the ISD 110 to vary as a lateral dimension of the vascular structure changes. In an example, a decrease in the lateral dimension in the vascular structure compresses the interconnected structure 122 thereby decreasing the lateral dimensions of the interconnected structure 122. Decreasing the lateral dimension of the interconnected structure 122 causes the cutting elements 118 to move radially inwardly since the cutting segments 116 are attached to and extend from the interconnected structure 122. As such, a decrease in the lateral dimensions of the vascular structure does not cause the cutting elements 118 to excessively cut the inner surface of the vascular structure that may otherwise cause excessive damage or cause the cutting segments to penetrate through the vascular structure. In an example, an increase in a lateral dimension in the vascular structure allows the interconnected structure 122 to expand thereby increasing the lateral dimensions of the interconnected structure 122. Again, increasing the lateral dimension of the interconnected structure 122 causes the cutting elements 118 to move radially outward relative to the catheter 102. As such, the cutting elements 118 continue to consistently scrape against the inner surface of the vascular structure even when the lateral dimension of the vascular structure increases.

The interconnected structure 122 formed by the linking segments 120 may exhibit any suitable shape. In an example, as illustrated, the interconnected structure 122 formed by the linking segments 120 may exhibit a generally conical shape exhibiting a diameter that increases in the distal direction. The generally conical shape of said interconnected structure 122 may facilitate decreasing or increasing the lateral dimension of the interconnected structure 122 as the ISD 110 is pulled in the vascular structure. The generally circular cross-sectional shape of the conical interconnected structure 122 may facilitate usage of the ISD 110 in the vascular structure since most vascular structures exhibit a generally circular cross-sectional shape. In an example, the interconnected structure 122 may exhibit a generally convex or concave funnel-like shape. Such funnel-like shapes of the interconnected structure 122 may exhibit some of the same benefits of as the conical shape interconnected structure 122. In an example, the interconnected structure 122 may exhibit a shape exhibiting a bulge between a proximal and distal end thereof which may facilitate increasing and decreasing a lateral dimension of the interconnected structure 122 when the ISD 110 moves distally (i.e., pushed) in the vascular structure.

The interconnected structure 122 may be atraumatic. That is, the interconnected structure 122 may be configured to prevent or at least inhibit damage to the vascular structure as the interconnected structure 122 abuts and moves relative to the vascular structure. The atraumatic interconnected structure 122 allows better control over damaging the vascular structure since, predominately, only the cutting elements 118 damage the vascular structure and variations in the size of the vascular structure (and the pressure applied from the interconnected structure 122 to the vascular structure as the lateral dimensions varies) does not significantly vary the damage caused to the vascular structure. The interconnected structure 122 may be configured to be atraumatic by rounding any edges and/or smoothing any surfaces that are likely to contact the vascular structure during use.

The linking segments 120 and the interconnected structure 122 formed thereby decreases the length of the cutting elements 118 compared to conventional mechanochemical ablation devices that do not include the linking segments 120. The decreased length of the cutting elements 118 allows better control over the cutting elements 118 during operation. For example, the decreased length of the cutting segments 116 allows the cutting elements 118 to apply a more consistent pressure against the vascular structure as the ISD 110 moves in the vascular structure. The decreased length of the cutting segments 116 may also minimize jumping of the cutting elements 118. For instance, during use, the cutting elements 118 may engage with (i.e., become jammed or stuck in) the vascular structure. In such instances, the cutting elements 118 may suddenly jump when disengaging with the vascular structure. The damage caused by the jumping cutting elements 118 is generally uncontrolled and may result in excessive or, more likely, little to no damage to the portions vascular structure that the cutting elements 118 jump. The likelihood that the cutting elements 118 jump and the distance that the cutting elements 118 jump decreases as the length of the cutting segments 116 is decreased.

The linking segments 120 may exhibit any suitable structure, for example, that allows the linking segments 120 to form the interconnected structure 122. In an example, the linking segments 120 may include one or more longitudinally extending segments 124. The longitudinally extending segments 124 are linking segments 120 that extend generally parallel to a longitudinal axis of the catheter 102 and/or the ISD 110 when the ISD 110 is in the collapsed configuration. The linking segments 120 may also include two or more branched segments 126 extending from the longitudinally extending segments. The branched segments 126 may extend from the longitudinally extending segments to other longitudinally extending segments 124 or other branched segments 126 thereby forming the interconnected structure 122.

The cutting segments 116 extend from the linking segments 120. In an example, as illustrated, the cutting segments 116 extend from the portion of the interconnected structure 122 that is configured to abut the vascular structure and from a location where two linking segments 120 intersect. It is noted that the cutting segments 116 do not necessarily extend from a location where two linking segments 120 intersect. For example, the cutting segments 116 may extend from a longitudinally extending segment 124. It is also noted that the cutting segments 116 may extend from portions of the interconnected structure 122 that are not configured to abut the vascular structure, such as when the interconnected structure 122 defines a bulge.

The cutting segments 116 of the ISD 110 exhibit a length measured from a proximal-most portion 128 of the cutting segment 116 to and a distalmost portion 130 of the cutting segment 116 in a direction that is parallel to a path that the cutting segments 116 extend (e.g., a generally parabolic path, as shown). The length of the cutting segments 116 may be about 0.5 mm to about 10 mm, such as in ranges of about 0.5 mm to about 1 mm, about 0.75 mm to about 1.5 mm, about 1 mm to about 2 mm, about 1.5 mm to about 2.5 mm, about 3 mm to about 4 mm, about 3.5 mm to about 4.5 mm, about 4 mm to about 5 mm, about 4.5 mm to about 6 mm, about 5 mm to about 7 mm, about 6 mm to about 8 mm, about 7 mm to about 9 mm, or about 8 mm to about 10 mm.

In an embodiment, the length of the cutting segments 116 may vary. For example, the lengths of the cutting segments 116 may vary by about 0.1 mm to about 5 mm, such as in ranges of about 0.1 mm to about 0.5 mm, about 0.25 mm to about 0.75 mm, about 0.5 mm to about 1 mm, about 0.75 mm to about 1.25 mm, about 1 mm to about 1.5 mm, about 1.25 mm to about 1.75 mm, about 1.5 mm to about 2 mm, about 1.75 mm to about 2.25 mm, about 2 mm to about 2.5 mm, about 2.25 mm to about 2.75 mm, about 2.5 mm to about 3 mm, about 2.75 mm to about 3.5 mm, about 3 mm to about 4 mm, about 3.5 mm to about 4.5 mm, or about 4 mm to about 5 mm. The varying lengths of the cutting segments 116 decrease the likelihood that the cutting segments 116 become entangled when the ISD 110 is in the collapsed configuration which may inhibit switching the ISD 110 from the collapsed configuration to the expanded configuration. For example, entanglement of the cutting segments 116 is most likely to occur when the distalmost portions 130 of the cutting segments 116 contact and overlap with each other. The varying lengths of the distalmost portions 130 offsets the distalmost portions 130 relative to each other, thereby decreasing the likelihood that the distalmost portions 130 of the cutting segments 116 contact and overlap with each other. The varying lengths of the cutting segments 116 also increases the number of cutting segments 116 that the ISD 110 may include. For instance, the likelihood that the cutting segments 116 becomes entangled also depends on the number of cutting segments 116. In particular, increasing the number of cutting segments 116 increases the likelihood of entanglement. However, the decreased likelihood of entanglement caused by the varying lengths of the cutting segments 116 allows the ISD 110 to include more cutting segments 116 while maintaining the likelihood of entanglement below a desired threshold. Varying the lengths of the cutting segments 116 also allows the cutting segments 116 to scrape against the vascular structure in different manners. For instance, varying the lengths of the cutting segments 116 varies the strain applied to the cutting segments 116 during use, with generally shorter cutting segments 116 exhibiting higher strains than longer cutting segments 116. The pressure applied by the cutting elements 118 to the vascular structure will vary as a function of the strain. The different pressures applied to the vascular structure may promote collapse of the vascular structure or otherwise provide a beneficial effect to the vascular structure. In an embodiment, two or more of the cutting segments 116 may exhibit the same length.

The ISD 110 may include any suitable number of cutting segments 116. For example, the ISD 110 may include 2, 3, 4, 5, 6, 7, 8, 9, 10, 11, 12, 13, 14, 15, 16, or greater than 16 cutting segments 116. Generally, increasing the number of cutting segments 116 increases the efficiency of the ISD 110. However, there is a threshold value above which adding more cutting segments 116 to the ISD 110 has no effect or negligible effect. As such, the number of cutting segments 116 of the ISD 110 may be selected to be at or below the threshold value. The threshold value may depend on a number of factors, including the procedure that the ISD 110 is being used in, the lateral dimensions of the vascular structure, the particular vascular structure that the cutting elements 118 are scraping, the number of cutting elements 118 on each cutting segment 116, and how the ISD 110 moves in the vascular structure (e.g., pulling, pushing, and/or rotating). The number of cutting segments 116 also affects the likelihood that cutting segments 116 become entangled with each other when the ISD 110 is in the collapsed state. As such, the number of cutting segments 116 may be selected to ensure that the likelihood that the cutting segments 116 become entangled remains below an acceptable percentage.

Each cutting segment 116 includes one or more cutting elements 118. The cutting elements 118 are the portions of the cutting segments 116 that are configured to abut the vascular structure and scrape or otherwise damage the vascular structure. In the illustrated embodiment, the cutting elements 118 are a sharpened point at the distalmost portion. It is noted that the cutting elements 118 are configured to gently damage the vascular structure thereby preventing or at least inhibiting the cutting elements 118 from causing excessive damage to the vascular structure. The gentle damaging of the vascular structure with the cutting elements 118 also minimizes the likelihood that the cutting elements 118 engage with the vascular structure.

FIG. 1C is a top plan view of a cutting element 118, according to an embodiment. The cutting element 118 includes a cutting surface 132. The cutting surface 132 may include a sharpened point and/or a sharpened edge (e.g., extending from the sharpened point). The cutting surface 132 may be formed using any suitable technique, such as grinding or laser cutting the cutting segment 116. The cutting surface 132 may extend at an angle θ relative to a longitudinal axis 134 of the cutting segment 116. The angle θ is the smallest angle extending between the cutting surface 132 and the longitudinal axis 134. The angle θ may be selected to be about 20° to about 90°, such as in ranges of about 30°to about 60°, about 20°to about 30°, about 25°to about 35°, about 30° to about 40°, about 35° to about 45°, about 40° to about 50°, about 45° to about 55°, about 50° to about 60°, about 55° to about 70°, about 65° to about 80°, or about 75° to about 90°. It is noted that the angle θ may be selected to be 30° or greater to prevent the cutting element 118 from excessively damaging the vascular structure since an angle θ that is less than 30° would make the cutting element 118 excessively sharp and allow the cutting element 118 to penetrate too deep into the vascular structure. That said, the angle θ may be selected to be about 20° to about 30°, for example, when deeper penetration is desired, which may depend on the application of the ISD 110. The angle θ is selected to be 60° or less to prevent the cutting element 118 from being too dull thereby inhibiting the cutting element 118 from even gently damaging the vascular structure. That said, the angle θ may be selected to be about 60° to about 90°, for example, when minimal damage to the vascular structure is desired. It is noted that at least one of the cutting elements 118 may include cutting surfaces 132 exhibiting a different angle θ than another cutting element 118 and/or at least two of the cutting elements 118 may include cutting surfaces 132 exhibiting the same angle θ.

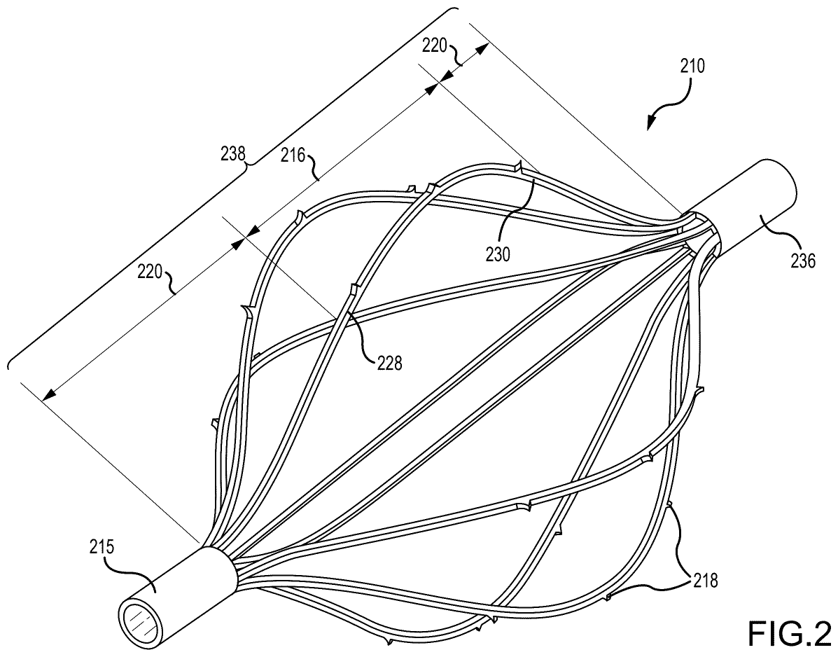

It is noted that the cutting elements 118 may include features other than or in addition to the cutting surface 132 that is configured to damage the vascular structure. In an example, the cutting elements 118 may include a roughened surface that is configured to rub against and damage the vascular structure as the ISD 110 moves relative to the vascular structure. In an example, the cutting segments 116 may include protrusions spaced from the distalmost end 130, similar to the cutting elements shown in FIGS. 2-4B.

In an embodiment, the ISD 110 exhibits a single piece construction. In such an embodiment, the ISD 110 may be formed by cutting a single piece of material to form the general features of the ISD 110, with optional post-cutting processing (e.g., grinding the cutting segments 116 to form the cutting surface 132). The single piece construction may facilitate manufacturing of the ISD 110 because the relatively small size of the ISD 110 and, in particular, the even smaller size of the individual components of the ISD 110 may make it difficult to attach the different features of the ISD 110 together. Further, some of the materials of the ISD 110 (e.g., nitinol) may be difficult to attach using welding or adhesives. That said, the ISD 110 may be formed from a plurality of distinct and separate pieces that are attached together, for example, using welds, adhesives, or any other suitable technique.

In an embodiment, at least a portion of the ISD 110 includes an R-phase nickel-titanium alloy (“R-phase alloy”). For example, the cutting segments 116 and, in some embodiments, the linking segments 120 may include an R-phase alloy. R-phase alloy is not used in at least some conventional catheter devices because R-phase alloy does not exhibit the superelasticity or shape memory effect of commonly used nickel-titanium alloys exhibiting austenitic phase at body temperature (“austenitic nitinol”). It is noted that the generic term “nitinol” generally refers to austenitic nitinol (especially if the nickel-titanium alloy is being used for its elasticity and/or shape memory properties) and that austenitic nitinol is commonly used in conventional catheter devices. However, unlike austenitic nitinol, the R-phase alloy generally exhibits a non-linear stress-strain curve at strains below roughly 1%. Instead, the R-phase alloy may be processed to exhibit a relatively shallow (e.g., flat) stress-strain curve at strains less than 0.7%. For example, the stress required to strain the R-phase alloy to about 0.7% is relatively negligible (e.g., about 33% of the stress required to strain the same material in austenitic phase to 0.7%). This means that the cutting segments 116 and, in particular, the cutting elements 118 apply a low and relatively consistent pressure (i.e., load or stress) against the vascular structure even as variations in the vascular structure cause the strain in the cutting segments 116 to vary. In other words, forming the cutting segments 116 from the R-phase alloy allows the cutting segments 116 to apply a generally low and consistent pressure to the vascular structure even if variations in the vascular structure cause variations in the strain applied to the cutting segments 116. Forming the cutting segments 116 from other materials, such as austenitic nitinol, may cause the cutting segments 116 to apply an inconsistent pressure to the vascular structure as the strain of the cutting segments 116 changes wherein resulting in excessive damage or negligible damage to the vascular structure. The inconsistent pressure applied to the vascular structure by the cutting segments 116 also increases the likelihood that the cutting elements 118 engages the vascular structure and jumps when becoming disengaged thereby resulting in inconsistent damage to the vascular structure.

It is noted that the material may be processed in such a way (e.g., by controlling the percent cold work, annealing temperature, and annealing time used to form the material including the R-phase alloy) that the slope of the stress-strain curve of the R-phase alloy increases rapidly at strains greater than 0.7%. As such, the ISD 110 including the R-phase alloy may be configured to maintain strain therein below 0.7% during normal operating conditions. In an example, as previously discussed, the linking segments 120 are configured to abut the vascular structure and vary the lateral dimensions thereof as the lateral dimensions of the vascular structure change which minimizes strain applied to the cutting segments 116. In another example, the strain of the cutting segments 116 depends on the length of the cutting segments 116, with smaller cutting segments 116 exhibiting higher strains than longer cutting segments 116. As such, the cutting segments 116 may exhibit a length that is sufficient to maintain a strain applied thereto under 1% and, more particularly, under 0.7% during normal operation conditions.

It is noted that the ISD 110 may include material other than, or in addition to, the R-phase alloy. For example, the ISD 110 (including the cutting segments 116) may include austenitic nitinol, stainless steel, other biocompatible metals, biocompatible polymers, composites, any other suitable material, or combinations of any of the foregoing. In an example, the cutting segments 116 may include a material that is different than the linking segments 120 or the proximal end 115 since variations in the strain in the linking segments 120 and the proximal end 115 have negligible effect on the damage of the vascular structure.

The ISD 110 may exhibit a maximum lateral dimension. As used herein, the maximum lateral dimension is the maximum lateral dimension (e.g., maximum diameter) of the portions ISD 110 formed by the linking segments 120 (e.g., the interconnected structure 122) when the ISD 110 is in the expanded configuration and there is no object (e.g., vascular structure) that prevents the ISD 110 from expanding. The maximum lateral dimension of the ISD 110 may be selected to be about 5 mm or greater, such as in ranges of about 5 mm to about 10 mm, about 7.5 mm to about 12.5 mm, about 10 mm to about 15 mm, about 12.5 mm to about 17.5 mm, about 15 mm to about 20 mm, about 17.5 mm to about 22.5 mm, about 20 mm to about 25 mm, about 22.5 mm to about 27.5 mm, about 25 mm to about 30 mm, or greater than 30 mm. The maximum lateral dimension may be selected to be greater than the maximum lateral dimension of the vascular structure that the ISD 110 is configured to gently damage thereby ensuring that the linking segments 120 maintain contact with the vascular structure during use.

The ISD 110 may exhibit a maximum length. As used herein, the maximum length is the length of the ISD 110 in the expanded configuration measured from the proximal most portion of the proximal end 115 to the distalmost portion 130 of the longest cutting segment 116 measured parallel to the longitudinal axis of the catheter to which the ISD 110 is attached or configured to be attached. The maximum length of the ISD 110 may be selected to be about 5 mm or greater, such as in ranges of about 5 mm to about 10 mm, about 7.5 mm to about 12.5 mm, about 10 mm to about 15 mm, about 12.5 mm to about 17.5 mm, about 15 mm to about 20 mm, about 17.5 mm to about 22.5 mm, about 20 mm to about 25 mm, about 22.5 mm to about 27.5 mm, about 25 mm to about 30 mm, about 27.5 mm to about 32.5 mm, about 30 mm to about 35 mm, about 32.5 mm to about 37.5 mm, about 35 mm to about 40 mm, or 40 mm or greater.

It is noted that the ISDs disclosed herein may exhibit a shape that is different than the ISD 110 shown in FIGS. 1A-1C. For example, FIG. 2 is an isometric view of an ISD 210, according to an embodiment. Except as otherwise disclosed herein, the ISD 210 is the same as or substantially similar to any of the ISDs disclosed herein. For example, the ISD 210 includes a proximal end 215 that is configured to be attached to a catheter (not shown). The ISD 210 also includes a plurality of cutting segments 216 having one or more cutting elements 218 and a plurality of linking segments 220.

The ISD 210 includes a distal end 236 on an opposing side of the ISD 210 from the proximal end 215. The distal end 236 is configured to be attached to a catheter and, in particular, a distal end region of the catheter. For example, the proximal end 215 may be configured to be attached to a first portion of the distal end region of the catheter and the distal end 236 may be configured to be attached to a second portion of the distal end region of the catheter that is positioned distally from the first portion. The distal end 236 may be attached to the catheter using any of the techniques disclosed herein or known in the art.

During use, the proximal and distal ends 215, 236 are configured to move relative to each other. The movement of the proximal and distal ends 215, 236 may facilitate switching the ISD 210 from the collapsed to the expanded configuration and may control, in part, the maximum lateral dimension of the ISD 210. For example, the catheter to which the proximal and distal ends 215, 236 are attached may include an inner lumen and an outer lumen that slides along the inner lumen. The proximal end 215 may be attached to the outer lumen (i.e., the outer lumen includes the first portion) and the distal end 236 may be attached to the inner lumen (i.e., the inner lumen includes the second portion). Sliding the outer lumen on the inner lumen in a distal direction decreases the distance between the proximal and distal ends 215, 236 thereby causing the elongated members 238 including the cutting segments 216 and the linking segments 220 to bow outwardly thereby increasing the maximum lateral dimension of the ISD 210 and causing the ISD 210 to be in the expanded configuration. Sliding the outer lumen on the inner lumen in a proximal direction increases the distance between the proximal and distal ends 215, 236 thereby causing the elongated members 238 to straighten thereby decreasing the maximum lateral dimension of the ISD 210 and causing the ISD 210 to be in the collapsed configuration.

The ISD 210 includes a plurality of elongated members 238 extending between the proximal and distal ends 215, 236. Each elongated member 238 includes one of the cutting segments 216 and two linking segments 220. One of the linking segments 220 extends from a proximal-most portion 228 of the cutting segment 216 to the proximal end 215 thereby linking the cutting segment 216 to the proximal end 215. The other one of the linking segments 220 extends from a distalmost portion 230 of the cutting segment 216 to the distal end 236 thereby linking the cutting segment 216 to the distal end 236. The ISD 210 may be more rigid than the ISD 110, illustrated in FIGS. 1A and 1B, since both ends of the elongated members 238 are secured to the catheter via the proximal and distal ends 215, 236.

In an embodiment, the cutting elements 218 of the ISD 210 are protrusions extending from the cutting segments 216. The damage caused by the cutting elements 218 is limited by the height of the cutting elements 218. As such, the cutting elements 218 may exhibit a height that is selected to prevent excessive damage to the vascular structure (e.g., prevents over penetration of the cutting elements 218 into the vascular structure). For example, the cutting elements 218 may exhibit a maximum height, measured from the portions of the cutting segment 216 thereabout in a radial direction (relative to the catheter, of about 50 μm to about 500 μm, such as in ranges of about 50 μm to about 100 μm, about 75 μm to about 150 μm, about 100 μm to about 200 μm, about 150 μm to about 250 μm, about 200 μm to about 300 μm, about 250 μm to about 350 μm, about 300 μm to about 400 μm, about 350 μm to about 450 μm, or about 400 μm to about 500 μm.

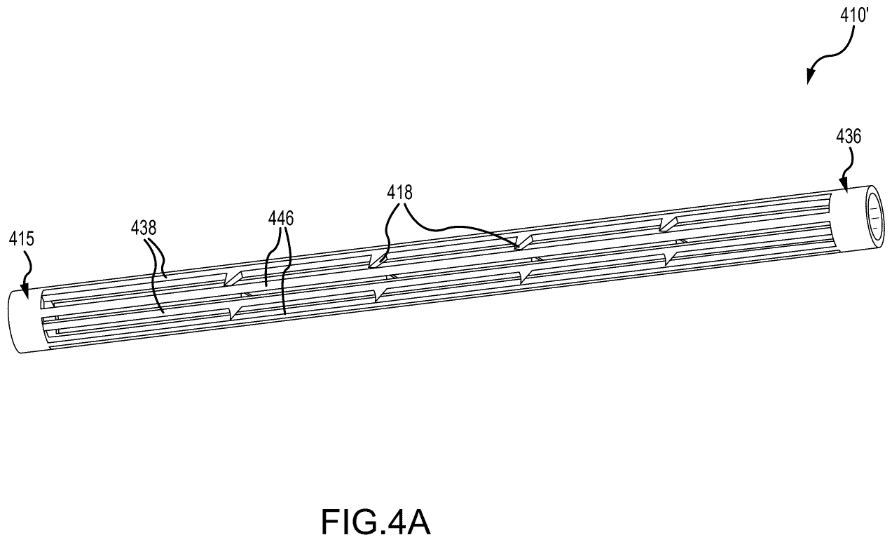

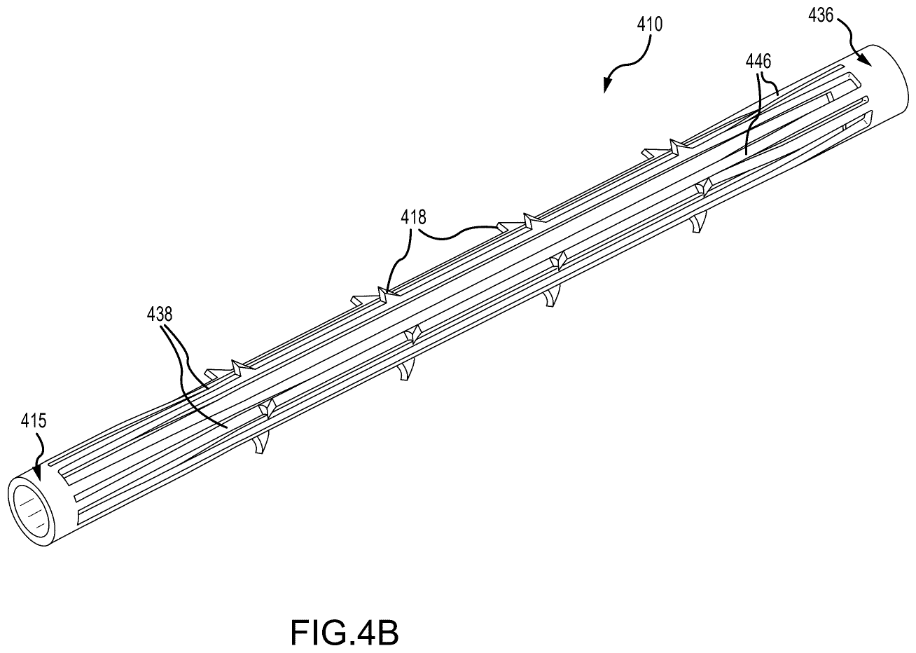

The ISD 210 may be formed using any suitable technique. Due to the limited size of the cutting elements 218, it may be difficult to attach the cutting elements 218 to rest of the cutting segment 216. As such, in some embodiments, the ISD 210 may include the cutting elements 218 integrally formed (e.g., exhibit single construction) with the rest of the cutting segments 216. FIGS. 3A-4B illustrate methods that may be used to form ISDs including cutting elements integrally formed with the rest of the cutting segments. That said, in some embodiment, the cutting elements 218 may be distinct and separate from the cutting segments 216 and, thus, are attached to the rest of the cutting segments 216.

FIG. 3A is an isometric view of an unassembled ISD 310′, according to an embodiment. The unassembled ISD 310′ may be formed by providing a generally tubular structure and then cutting the features of the completed ISD 310 (shown in FIG. 3B) into the generally tubular structure. For example, the features formed in the generally tubular structure may include a proximal end 315, a distal end 336, and a plurality of elongated members 338.

The proximal and distal ends 315, 336 of the unassembled ISD 310′ may include a plurality of recesses 340 formed therein. Each of the recesses formed in the proximal and distal ends 315, 336 may exhibit a size that is configured to receive the elongated members 338 when the cutting elements 318 of the elongated members 338 are oriented generally radially outward. In an embodiment, each of the proximal and distal ends 315, 336 of the unassembled ISD 310′ include an outward side 342 and an inward side 344. The elongated members 338 may extend between the inward sides 344 of the proximal and distal ends 315 and the recesses 340 may extend inwardly from the outward side of the proximal and distal ends 315, 336.

The elongated members 338 include the cutting elements 318 (e.g., protrusions) formed thereon when the unassembled ISD 310′ is formed. Due to the generally tubular structure from which the elongated members 338 are formed, the cutting elements 318 cannot be initially formed as extending radially outward from the elongated members 338. Instead, the cutting elements 318 are initially formed as extending generally circumferentially from the elongated members 338.

After forming the unassembled ISD 310′, the elongated members 338 may be separated from the proximal and distal ends 315, 336. In the illustrated embodiment, separating the elongated members 338 from the proximal and distal ends 315, 336 may include cutting the unassembled ISD 310′ along dashed line A, shown in FIG. 3A. The elongated members 338 may be separate from the proximal and distal ends 315, 336 using any suitable technique, such as laser cutting.

After separating the elongated members 338 from the proximal and distal ends 315, 336, the proximal and distal ends 315, 336 may be oriented such that the recesses 340 of the proximal end 315 generally face the recesses 340 of the distal end 336. The elongated members 338 may then be oriented (e.g., rotated 90°) such that the cutting elements 318 are oriented to extend radially outward from the rest of the elongated members 338. The ends of the elongated members 338 may then be inserted into the recesses 340 of the proximal and distal ends 315, 336. With the elongated members 338 inserted into the recessed 340, the elongated members 338 may be attached to the proximal and distal ends 315, 336 using any suitable technique, such as with an adhesive, soldering, welding, or mechanical interlocking. Attaching the elongated members 338 to the proximal and distal ends 315, 336 forms the assembled ISD 310, which is shown in FIG. 3B (which is an isometric view of the assembled ISD 310).

FIGS. 4A and 4B are isometric views of an unassembled ISD 410′ and an assembled ISD 410, respectively, and illustrate a method of forming the assembled ISD 410, according to an embodiment. The unassembled ISD 410′ may be formed by providing a generally tubular structure and then cutting the features of the completed ISD 410 (shown in FIG. 4B) into the generally tubular structure. For example, the features formed in the generally tubular structure may include a proximal end 415, a distal end 436, and a plurality of elongated members 438. The cutting elements 418 of the elongated members 438 may be oriented circumferentially since the unassembled ISD 410′ is formed from a tubular structure.

The unassembled ISD 410′ may also include a plurality of secondary elongated members 446 formed therein. The secondary elongated members 446 are elongated members extending between the proximal and distal ends 415, 436 that do not include cutting elements formed therein. The secondary elongated members 446 may be positioned between each of the plurality of elongated members 438. In an example, the secondary elongated members 446 may be positioned immediately adjacent to the side of the elongated members 438 that does not include the cutting elements 418 extending therefrom.

The assembled ISD 410 is formed by at least rotating (e.g., via plastic or elastic deformations) each of the elongated members 438 about 90° such that the cutting elements 418 extend radially outward. After rotating the elongated members 438, the elongated members 438 may be attached to one of the secondary elongated members 446 using any suitable technique, such as with a weld. Attaching the rotated elongated members 438 to the adjacent secondary elongated member 446 fixes the elongated members 438 in the rotated position thereby maintaining the cutting element 418 oriented radially outward.

The rotated elongated members 438 may have a residual rotational stress that, without a counter stress, may cause the elongated members 438 and the secondary elongated member 446 to tilt to one side which may limit the ability of the cutting elements 418 to penetrate or otherwise damage the vascular structure. As such, in an embodiment, the secondary elongated members 446 may be rotated 90° in an opposite direction than the elongated members 438 before the elongated members 438 and the secondary elongated members 446 are attached together. This causes the secondary elongated members 446 to exhibit a residual rotational stress that is opposite the residual rotational stress of the elongated members 438 thereby preventing or limiting the elongated members 438 and the secondary elongated member 446 from tilting.

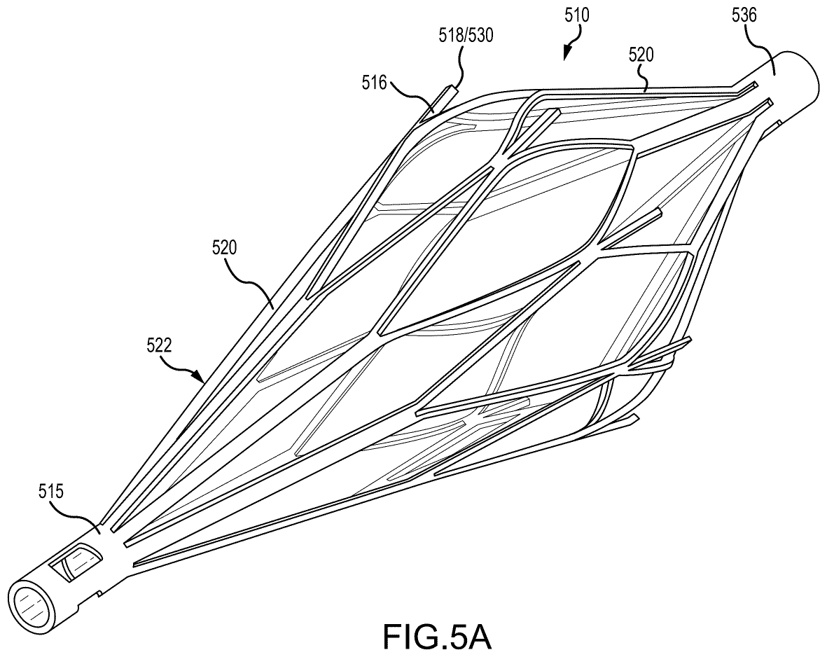

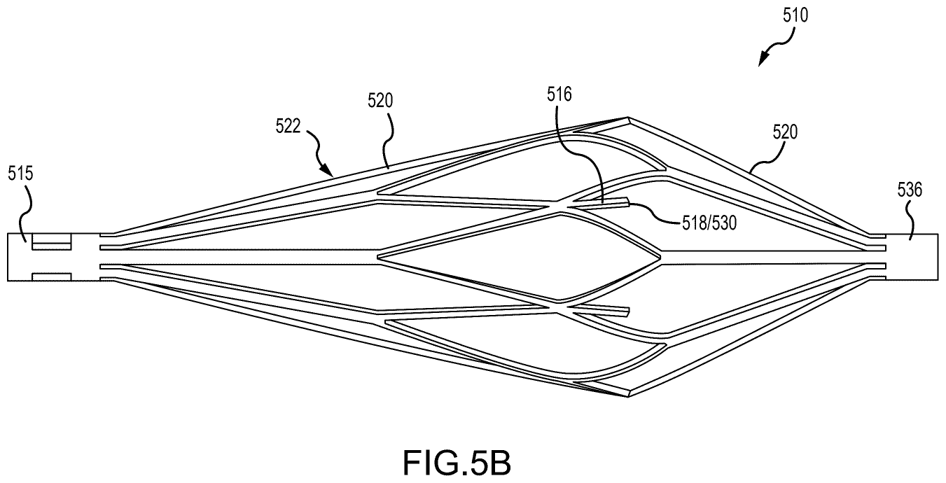

FIGS. 5A and 5B are an isometric and side plan view of an ISD 510, according to an embodiment. Except as otherwise disclosed herein, the ISD 510 is the same as or substantially similar to any of the ISDs disclosed herein. For example, the ISD 510 includes a proximal end 515, a distal end 536, a plurality of cutting elements 518, and a plurality of linking segments 520.

The linking segments 520 form an interconnected structure 522 extending from the proximal end 515 to the distal end 536. The interconnected structure 522 extending between the proximal and distal ends 515, 536 increases the rigidity of the interconnected structure 522 than if the interconnected structure 522 only extended from one of the proximal end 515 or the distal end 536. In an embodiment, as shown, the interconnected structure 522 may form a bulge between the proximal and distal ends 515, 536. The bulge of the interconnected structure 522 may exhibit the maximum lateral dimension of the interconnected structure 522. The bulge of the interconnected structure 522 may be configured to abut the vascular structure during use such that the bulge exhibits a maximum lateral dimension that corresponds to the maximum lateral dimension of the vascular structure. The maximum lateral dimension of the bulge may vary as the maximum lateral dimension of the vascular structure varies, as previously discussed.

The interconnected structure 522 may form any suitable shape. In an embodiment, as shown, the interconnected structure 522 may exhibit a generally circular cross-sectional shape which allows the interconnected structure 522 to exhibit a cross-sectional shape that generally corresponds to the cross-sectional shape of most vascular structures. In an embodiment, as shown, the interconnected structure 522 includes a proximal region located between the bulge and the proximal end 515 and a distal region located between the bulge and the distal end 536. In an example, the proximal and distal regions may each exhibit a generally conical shape, wherein the bases of each of the proximal and distal regions abut each other. In another example, one or both of the proximal region or the distal region of the interconnected structure 522 may exhibit a tapered shape other than a generally conical shape. In an example, the sizes (e.g., length) of the proximal and distal regions may be different.

The ISD 510 illustrated in FIGS. 5A and 5B are illustrated as being in the expanded configuration. The ISD 510 switches between the collapsed and expanded configurations by moving the proximal end 515 and the distal end 536 apart and closer together, respectively. The proximal and distal ends 515, 536 may move apart and closer together using the inner and outer lumens discussed with regards to FIG. 2 or using any other suitable technique. Switching the ISD 510 from the collapsed configuration to the expanded configuration increases a maximum lateral dimension of the interconnected structure 522. Switching the ISD 510 from the expanded configuration to the collapsed configuration decreases a maximum lateral dimension of the interconnected structure 522.

The cutting segments 516 each include one or more cutting elements 518. The cutting elements 518 may be the same as or substantially similar to any of the cutting elements 518 disclosed herein. In an embodiment, as shown, the cutting segments 516 and the cutting elements 518 are substantially similar to the cutting segments 116 and the cutting elements 118 illustrated in FIGS. 1A-1C. That is, the cutting segments 516 extend from the linking segments 520 (e.g., from or near the bulge of the interconnected structure 522) and the distalmost portion 530 of the cutting segments 516 include the cutting element 518 (i.e., the cutting surface 532). In an embodiment, the cutting segments 516 may include one or more protrusions (i.e., cutting elements 518) extending therefrom. In such an embodiment, the cutting segments 516 may form the bulge thereby allowing at least some of the protrusions to be present at or near the bulge. In other words, the cutting segments 516 may form part of the interconnected structure 522.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

What is claimed is:

1. An intravascular scraping device, comprising:
a proximal end configured to be attached to a catheter;
a plurality of cutting segments, each of the plurality of cutting segments including one or more cutting elements; and
a plurality of linking segments, at least some of the plurality of linking segments linking the plurality of cutting segments to the proximal end, at least some of the plurality of linking segments directly extending from the proximal end and at least some of the plurality of linking segments directly extending from the plurality of cutting segments generally towards the proximal end.

2. The intravascular scraping device of claim 1, wherein at least some of the plurality of linking segments are directly linked together.

3. The intravascular scraping device of claim 2, wherein the intravascular scraping device does not include a distal end configured to be attached to the catheter.

4. The intravascular scraping device of claim 2, wherein the intravascular scraping device includes a distal end configured to be attached to the catheter, some of the linking segments directly extending from each of:
the proximal end;
the distal end;
each of the plurality of cutting elements towards the proximal end; and
each of the plurality of cutting elements towards the distal end.

5. The intravascular scraping device of claim 2, wherein each of the plurality of cutting segments includes a proximal-most portion and a distalmost portion opposite the proximal-most portion, the proximal-most portion of each of the plurality of cutting segments is directly linked with one of the plurality of linking segments, the distal-most portion forming the cutting element.

6. The intravascular scraping device of claim 5, wherein each of the plurality of cutting segments exhibits a length measured from the proximal-most portion to the distalmost portion, the length of at least one of the plurality of cutting segments is different than a length of at least one other one of the plurality of cutting segments.

7. The intravascular scraping device of claim 1, wherein some of the plurality of linking segments link each of the plurality of cutting segments to a distal end that is spaced from the proximal end, the distal end configured to be attached to the catheter, some of the plurality of linking segments directly extending from the distal end.

8. The intravascular scraping device of claim 6, further comprising a plurality of elongated members extending from the proximal end to the distal end, each of the plurality of elongated members includes one of the plurality of cutting segments and two of the plurality of linking segments;
wherein, for each of the plurality of elongated members, one of the two linking segments extends from the cutting segment to the proximal end and another of the two linking segments extends from the cutting segment to the distal end.

9. The intravascular scraping device of claim 8, wherein:
each of the proximal end and the distal end define a plurality of recesses; and
each of the plurality of elongated are positioned in one of the plurality of recesses of the proximal end and one of the plurality of recesses of the distal end and attached to the proximal end and the distal end.

10. The intravascular scraping device of claim 8, further comprising a plurality of secondary elongated members extending between the proximal end and the distal end; and
wherein a portion of each of the plurality of elongated members are rotated and attached to an adjacent one of the plurality of secondary elongated member.

11. The intravascular scraping device of claim 10, wherein each of the second elongated members do not include a cutting element.

12. The intravascular scraping device of claim 1, wherein the plurality of cutting segments include an R-phase nickel-titanium alloy.

13. The intravascular scraping device of claim 12, wherein the plurality of linking segments includes an R-phase nickel-titanium alloy.

14. The intravascular scraping device of claim 1, wherein the plurality of linking segments include:
a longitudinal linking segment generally oriented parallel to a longitudinal axis of the intravascular scraping device when the intravascular scraping device is in a collapsed state; and
two or more branched linking segments extending from one end of the longitudinal linking segment.

15. A catheter, comprising:
a central catheter including a distal end region; and
an intravascular scraping device including:
a proximal end configured to be attached to a first portion of the distal end region;
a plurality of cutting segments, each of the plurality of cutting segments including one or more cutting elements; and
a plurality of linking segments, at least some of the plurality of linking segments linking the plurality of cutting segments to the proximal end, at least some of the plurality of linking segments directly extending from the proximal end and at least some of the plurality of linking segments directly extending from the plurality of cutting segments generally towards the proximal end.

16. The catheter of claim 15, wherein the intravascular scraping device includes a distal end and the distal end is attached to a second portion of the distal end region, the second portion is distally spaced from the first portion.

17. The catheter of claim 15, wherein the central catheter includes an interior passageway and the distal end region includes one or more holes in fluid communication with the interior passageway.

18. A method of forming an intravascular scraping device, the method comprising:
providing at least one generally cylindrical tube; and
cutting the generally cylindrical tube to form at least:
a proximal end configured to be attached to a catheter;
a plurality of cutting segments, each of the plurality of cutting segments including one or more cutting elements; and
a plurality of linking segments linking each of the plurality of cutting segments to at least the proximal end, at least some of the plurality of linking segments directly extending from the proximal end and at least some of the plurality of linking segments directly extending from the plurality of cutting segments generally towards the proximal end.

19. The method of claim 18, wherein:
providing the at least one generally cylindrical tube includes providing a single generally cylindrical tube;
cutting the generally cylindrical tube includes cutting the generally cylindrical tube to form:
the proximal end;
the plurality of cutting segments, the one or more cutting elements of each of the plurality of cutting segments oriented generally circumferentially; and
the plurality of linking segments; and
further comprising:
separating the plurality of cutting segments and the plurality of linking segments from the proximal end;
rotating the plurality of cutting segments to orient the one or more cutting element radially outward; and
attaching the plurality of cutting segments and the plurality of linking segments to the proximal end.

20. The method of claim 19, wherein:
cutting the generally cylindrical tube to form the proximal end includes forming a plurality of recesses in the proximal end; and
attaching the plurality of cutting segments and the plurality of linking segments to the proximal end includes positioning each of the plurality of linking segments in one of the plurality of recesses.

21. The method of claim 18, wherein:
providing that at least one generally cylindrical tube includes providing a single generally cylindrical tube;
cutting the generally cylindrical tube includes cutting the generally cylindrical tube to form:
the proximal end;
a plurality of elongated members, each of the plurality of elongated members include a cutting segment including one or more cutting segments oriented generally circumferentially and at least one linking segment extending between the proximal end and the cutting element; and
a plurality of secondary elongated members extending from the proximal end; and
further comprising:
rotating at least a portion of the plurality of elongated members to orient the one or more cutting segments radially outward; and
attaching each of the rotated plurality of elongated members to an adjacent one of the plurality of secondary elongated members.
